(19) United States
(12) Patent Application Publication
(10) Pub. No.: US 2011/0028812 A1
(43) Pub. Date: Feb. 3, 2011

(54) METHOD FOR SPECTROPHOTOMETRIC BLOOD OXYGENATION MONITORING

(75) Inventor: Paul Benni, Acton, MA (US)

Correspondence Address: O''Shea Getz P.C., 1500 MAIN ST. SUITE 912, SPRINGFIELD, MA, 01115, US

(73) Assignee: CAS MEDICAL SYSTEMS, INC., Branford, CT (US)

(21) Appl. No.: 12/851,977

(22) Filed: Aug. 6, 2010

Related U.S. Application Data
(63) Continuation-in-part of application No. PCT/US09/33543, filed on Feb. 9, 2009.
(60) Provisional application No. 61/027,056, filed on Feb. 8, 2008.

(51) Int. Cl. A61B 5/1455

(52) U.S. Cl. 600/323

(57) ABSTRACT

A method and apparatus for non-invasively determining a blood oxygenation level within a subject's tissue is provided. The method includes the steps of: a) providing a spectrophotometric sensor operable to transmit light into the subject's tissue, and to sense the light; b) inputting into the sensor at least one of the subject's age, weight, brain development, and head size; c) spectrophotometrically sensing the subject's tissue along a plurality of wavelengths using the sensor, and producing signal data from sensing the subject's tissue; and d) processing the signal data utilizing the at least one of the subject's age, weight, brain development, and head size, to determine the blood oxygen saturation level within the subject's tissue using a difference in attenuation between the wavelengths.

This application is a continuation-in-part of PCT Patent Application No. PCT/US09/33543 filed Feb. 9, 2009, which claims priority benefits under 35 U.S.C. §119(e) of U.S. Provisional Patent Application No. 61/027,056 filed Feb. 8, 2008, the disclosures of which are herein incorporated by reference.

This invention was made with Government support under Contract No. 2R44NS045488-02 awarded by the Department of Health & Human Services. The Government has certain rights in the invention.

### BACKGROUND OF THE INVENTION

1. Technical Field

This invention relates to methods for non-invasively determining biological tissue oxygenation in general, and to non-invasive methods utilizing near-infrared spectroscopy (NIRS) techniques for determining the same in particular.

2. Background Information

U.S. Pat. No. 6,456,862 and U.S. Pat. No. 7,072,701, both assigned to the assignee of the present application and both hereby incorporated by reference, disclose methods for spectrophotometric blood oxygenation monitoring. Oxygen saturation within blood is defined as:

$${O}_{2} saturation \% = \frac{Hb {O}_{2}}{( Hb {O}_{2} + Hb )} * 100 \% \qquad ( Eqn . 1 )$$

These methods, and others known within the prior art, utilize variants of the Beer-Lambert law to account for optical attenuation in tissue at a particular wavelength. Relative concentrations of oxyhemoglobin (HbO₂) and deoxyhemoglobin (Hb), and therefore oxygenation levels, within a tissue sample are determinable using changes in optical attenuation:

$$Δ {A}_{λ} = - {log ( \frac{{I}_{t 2}}{{I}_{t 1}} )}_{λ} {α}_{λ} * Δ C * d * {B}_{λ} \qquad ( Eqn . 2 )$$

wherein “A_{λ}” represents the optical attenuation in tissue at a particular wavelength λ (units: optical density or OD); “I” represents the incident light intensity (units: W/cm²); “α_{λ}” represents the wavelength dependent absorption coefficient of the chromophore (units: OD*cm⁻¹*μM⁻¹); “C” represents the concentration of chromophore (units: μM); “d” represents the light source to detector (optode) separation distance (units: cm); and “B_{λ}” represents the wavelength dependent light scattering differential pathlength factor (unitless)

To non-invasively determine oxygen saturation within tissue accurately, it is necessary to account for the optical properties (e.g., absorption coefficients or optical densities) of the tissue being interrogated. In some instances, the absorption coefficients or optical densities for the tissue components that create background light absorption and scattering can be assumed to be relatively constant over a selected wavelength range. The graph shown in FIG. 1, which includes tissue data plotted relative to a y-axis of values representative of absorption coefficient values and an x-axis of wavelength values, illustrates such an instance. The aforesaid constant value assumption is reasonable in a test population where all of the subjects have approximately the same tissue optical properties; e.g., skin pigmentation, muscle and bone density, etc. A tissue interrogation method that relies upon such an assumption may be described as being wavelength independent within the selected wavelength range and subject independent. The same assumption is not reasonable, however, in a population of subjects having a wide spectrum of tissue optical properties (e.g., a range of significantly different skin pigmentations from very light to very dark) unless consideration for the wide spectrum of tissue optical properties is provided otherwise.

What is needed, therefore, is a method for non-invasively determining the level of oxygen saturation within biological tissue that accounts for optical influences from the specific tissue through which the light signal passes.

### DISCLOSURE OF THE INVENTION

A method and apparatus for non-invasively determining the blood oxygen saturation level within a subject's tissue is provided. According to one aspect, a method for non-invasively determining a blood oxygenation level within a subject's tissue is provided that comprises the steps of: a) providing a spectrophotometric sensor operable to transmit light into the subject's tissue, and to sense the light; b) inputting into the sensor at least one of the subject's age, weight, brain development, and head size; c) spectrophotometrically sensing the subject's tissue along a plurality of wavelengths using the sensor, and producing signal data from sensing the subject's tissue; and d) processing the signal data utilizing the at least one of the subject's age, weight, brain development, and head size, to determine the blood oxygen saturation level within the subject's tissue using a difference in attenuation between the wavelengths.

According to another aspect, an apparatus for non-invasively determining a blood oxygenation level within a subject's tissue is provided having a sensor that includes one or more transducer portions and a processor portion. Each of the one or more transducer portions includes at least one light source and at least one light detector. The light source is operable to transmit light along a plurality of wavelengths into the subject's tissue, and the light detector is operable to detect light along the wavelengths traveling through the subject's tissue. Each of the transducer portions is operable to produce signal data representative of the light sensed within the subject's tissue. The processor portion is operably connected to the one or more transducer portions, and is adapted to receive input of at least one of the subject's age, weight, brain development, and head size. The processor portion is adapted to process the signal data utilizing at least one of the subject's age, weight, brain development, and head size, to determine the blood oxygen saturation level within the subject's tissue using a difference in attenuation between the wavelengths.

These and other objects, features, and advantages of the present invention method and apparatus will become apparent in light of the detailed description of the invention provided below and the accompanying drawings. The methodology and apparatus described below constitute a preferred embodiment of the underlying invention and do not, therefore, constitute all aspects of the invention that will or may become apparent by one of skill in the art after consideration of the invention disclosed overall herein.

### BRIEF DESCRIPTION OF THE DRAWINGS

FIG. 1 is a graph diagrammatically illustrating tissue data plotted relative to a y-axis of values representative of absorption coefficient values, and an x-axis of wavelength values.

FIG. 2 is a diagrammatic representation of a NIRS sensor.

FIG. 3 is a diagrammatic representation of a NIRS sensor placed on a subject's head.

FIG. 4 is a diagrammatic view of a NIRS sensor.

FIG. 5 is a graph having values diagrammatically representative of subject-specific calibration coefficients plotted along a y-axis, TOP index values plotted along an x-axis, and data representative of deoxyhemoglobin values and oxyhemoglobin values plotted therebetween with best-fit curves applied thereto.

FIG. 6 is a flow chart illustrating steps according to one aspect of the present invention.

FIG. 7 is a graph illustrating the relationship between calibration values and subject weight for pediatric subjects.

FIG. 8 is a diagrammatic graph illustrating the difference in SctO2 determined using a NIRS sensor that does not account for subject weight, and SctO2 determined by invasive blood sample along the y-axis, versus subject weight along the x-axis.

FIG. 9 is a diagrammatic graph illustrating the difference in SctO2 determined using a NIRS sensor that does account for subject weight, and SctO2 determined by invasive blood sample along the y-axis, versus subject weight along the x-axis.

FIG. 10 is a diagrammatic graph illustrating the difference in SctO2 determined using a NIRS sensor that does not account for subject age, and SctO2 determined by invasive blood sample along the y-axis, versus subject age along the x-axis.

FIG. 11 is a diagrammatic graph illustrating the difference in SctO2 determined using a NIRS sensor that does account for subject age, and SctO2 determined by invasive blood sample along the y-axis, versus subject age along the x-axis.

FIG. 12 is a diagrammatic view of a NIRS sensor disposed in a planar position and a flexed position in phantom.

FIG. 13 is a diagrammatic illustration of a transducer housing disposed on two different size 60 degree ellipses, which ellipses are representative of subject “heads”.

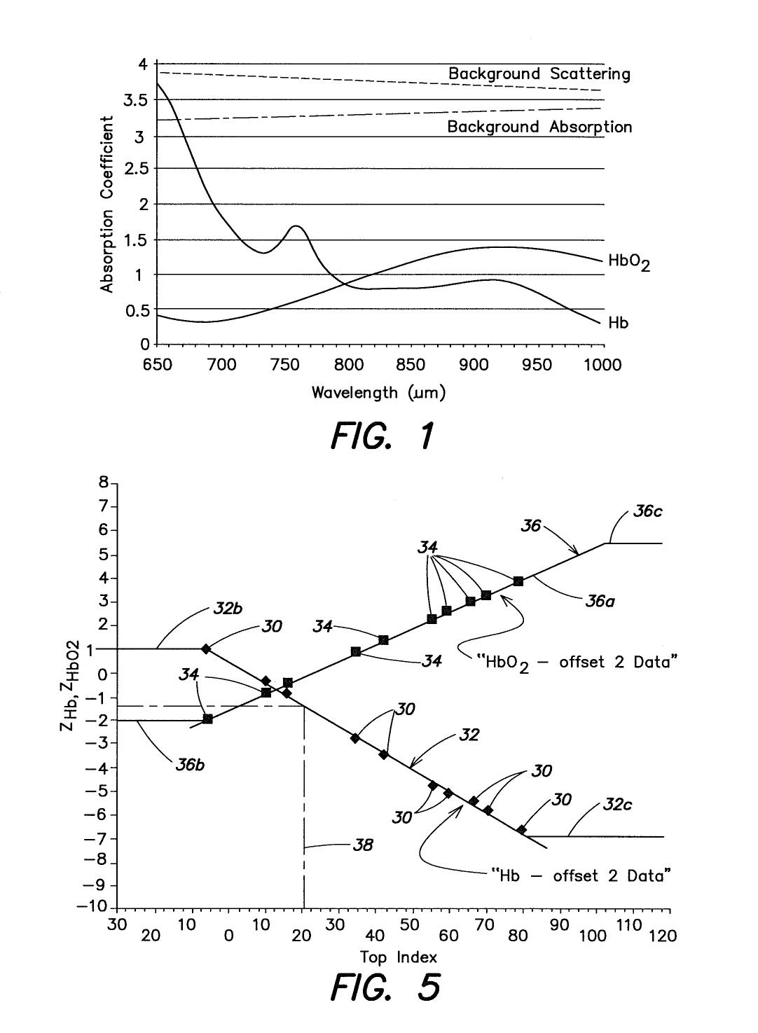

### DETAILED DESCRIPTION THE INVENTION

The present method of, and apparatus for, non-invasively determining the blood oxygen saturation level within a subject's tissue is provided that utilizes a near infrared spectrophotometric (NIRS) sensor that includes a transducer capable of transmitting a light signal into the tissue of a subject and sensing the light signal once it has passed through the tissue via transmittance or reflectance. The present method and apparatus can be used with a variety of NIRS sensors, and is not therefore limited to any particular NIRS sensor.

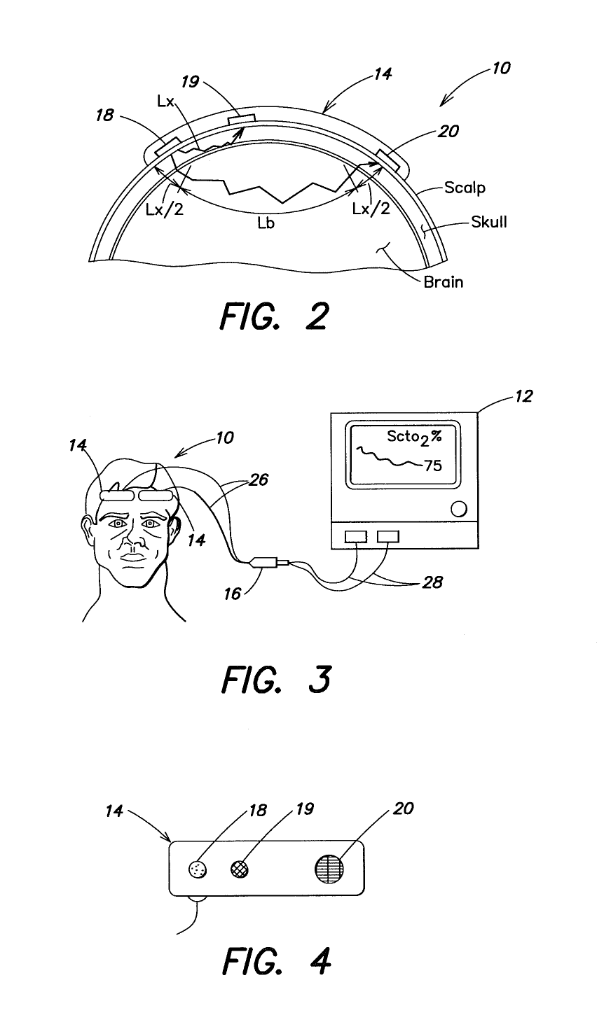

Referring to FIGS. 2-4, an example of an acceptable NIRS sensor includes a transducer portion 10 and processor portion 12. The transducer portion 10 includes an assembly housing 14 and a connector housing 16. The assembly housing 14, which is a flexible structure that can be attached directly to a subject's body, includes one or more light sources 18 and light detectors 19, 20. A disposable adhesive envelope or pad is preferably used for mounting the assembly housing 14 easily and securely to the subject's skin. Light sources selectively emit light signals of known but different wavelengths through a prism assembly. The light sources 18 are preferably laser diodes that emit light at a narrow spectral bandwidth at predetermined wavelengths. The laser diodes may be mounted remotely from the assembly housing 14; e.g., in the connector housing 16 or within the processor portion 12. In these embodiments, a fiber optic light guide is optically interfaced with the laser diodes and the prism assembly that is disposed within the assembly housing 14. In other embodiments, the light sources 18 are mounted within the assembly housing 14. A first connector cable 26 connects the assembly housing 14 to the connector housing 16 and a second connector cable 28 connects the connector housing 16 to the processor portion 12. The light detectors 19, 20 each include one or more photodiodes. The photodiodes are also operably connected to the processor portion 12 via the first and second connector cables 26, 28. Other examples of acceptable NIRS sensors are described in PCT Patent Publication No. WO 07/048,039 filed on Oct. 18, 2006 which application is commonly assigned to the assignee of the present application and which is hereby incorporated by reference in its entirety.

The processor portion 12 includes a processor for processing light intensity signals associated with the light sources 18 and the light detectors 19, 20 as described herein. A person of skill in the art will recognize that the processor may assume various forms (e.g., digital signal processor, analog device, etc.) capable of performing the functions described herein. The processor utilizes an algorithm that characterizes a change in attenuation as a function of the difference in attenuation between different wavelengths. The algorithm accounts for the effects of pathlength and parameter “E”, which represents energy losses, (“G”) due to light scattering within tissue, other background absorption losses (“F”) from biological compounds, and other unknown losses (“N”) including measuring apparatus variability (E=G+F+N). As will be discussed below, the parameter “E” reflects energy losses not specific to the subject being tested with a calibrated sensor (i.e., “subject-independent”).

The absorption A_{bλ} detected from the deep light detector 20 includes attenuation and energy losses from both the deep and shallow tissue, while the absorption A_{xλ} detected from the shallow light detector 19 includes attenuation and energy losses from shallow tissue. Absorptions A_{bλ} and A_{xλ} can be expressed in the form of Equation 3 and Equation 4:

$$\begin{matrix}{A}_{b λ} = - {log ( \frac{{I}_{b}}{{I}_{o}} )}_{λ} = {α}_{λ} * {C}_{b} * {L}_{b} + {α}_{λ} * {C}_{x} * {L}_{x} + {E}_{λ} & ( Eqn . 3 ) \\ {A}_{x λ} = - {log ( \frac{{I}_{x}}{{I}_{o}} )}_{λ} = {α}_{λ} * {C}_{x} * {L}_{x} + {E}_{x λ} & ( Eqn . 4 )\end{matrix}$$

In some applications (e.g., infants), a single light detector may be used, in which case Equation 5 is used:

A_{bλ}=−log(I_{b}/Iₒ)λ=α_{λ}*C_{b}*L_{b}+E_{λ} (Eqn 5)

If both the deep and shallow detectors are used, then substituting Equation 4 into Equation 3 yields A′λ, which represents attenuation and energy loss from deep tissue only:

A′_{λ}=A_{bλ}−A_{xλ}=α_{λ}*C_{b}*L_{b}+(E_{λ}−E_{xλ}) (Eqn.6)

From Equation 5 or Equation 6, L is the effective pathlength of the photon traveling through the deep tissue and A′₁ and A′₂ represent light attenuation at two different wavelengths to determine differential wavelength light attenuation ΔA′₁₂:

A′₁−A′₂=ΔA′₁₂ (Eqn.7)

Substituting Equation 5 or 6 into Equation 7 for A′₁ and A′₂, ΔA′₁₂ can be expressed as:

ΔA′₁₂=α_{λ12}*C_{b}*L_{b}+ΔE′₁₂ (Eqn.8)

and Equation 8 can be rewritten in expanded form:

ΔA′₁₂= [symbol] (αᵣ₁−αᵣ₂[Hb]_{b}+(αₒ₁−αₒ₂)[HbO₂]_{b} [symbol] L_{b}+(E′₁−E′₂)=(Δαᵣ₁₂*[Hb]_{b}*L_{b})+(Δαₒ₁₂*[HbO₂]_{b}*L_{b})ΔE′₁₂ (Eqn.9)

where:

(Δαᵣ₁₂*[Hb]_{b}*L_{b}) represents the attenuation attributable to Hb; and

(Δαₒ₁₂*[HbO₂]_{b}*L_{b}) represents the attenuation attributable to HbO₂; and

ΔE′₁₂ represents energy losses due to light scattering within tissue, other background absorption losses from biological compounds, and other unknown losses including measuring apparatus variability.

The multivariate form of Equation 9 is used to determine [HbO₂]_{b} and [Hb]_{b} with three different wavelengths:

$$[ \begin{matrix}Δ {A}_{12}^{′} & Δ {E}_{12}^{′} \\ Δ {A}_{13}^{′} & Δ {E}_{13}^{′}\end{matrix} ] {( {L}_{b} )}^{- 1} = [ \begin{matrix}Δ {α}_{r 12} & {Δα}_{o 12} \\ {Δα}_{r 13} & {Δα}_{013}\end{matrix} ] [ \begin{matrix}{[ Hb ]}_{b} \\ {[ Hb {O}_{2} ]}_{b}\end{matrix} ] \qquad ( Eqn . 10 )$$

Rearranging and solving for [HbO₂]_{b} and [Hb]_{b}, simplifying the Δαmatrix into [Δα′]:

$${[ \begin{matrix}Δ {A}_{12}^{′} \\ Δ {A}_{13}^{′}\end{matrix} ] [ {Δα}^{′} ]}^{- 1} {( {L}_{b} )}^{- 1} - {[ \begin{matrix}Δ {E}_{12}^{′} \\ Δ {E}_{13}^{′}\end{matrix} ] [ {Δα}^{′} ]}^{- 1} {( {L}_{b} )}^{- 1} = [ \begin{matrix}{[ Hb ]}_{b} \\ {[ Hb {O}_{2} ]}_{b}\end{matrix} ] \qquad ( Eqn . 11 )$$

Then combined matrices [ΔA′][Δα′]⁻¹=[A_{c}] and [ΔE][Δα′]^{-1=[Ψ}_{c}]:

$$[ \begin{matrix}{A}_{Hb} \\ {A}_{{HbO}_{2}}\end{matrix} ] {( {L}_{b} )}^{- 1} - [ \begin{matrix}{Ψ}_{Hb} \\ {Ψ}_{{HbO}_{2}}\end{matrix} ] {( {L}_{b} )}^{- 1} = [ \begin{matrix}{[ Hb ]}_{b} \\ {[ Hb {O}_{2} ]}_{b}\end{matrix} ] \qquad ( Eqn . 12 )$$

The parameters A_{Hb} and A_{HbO2} represent the product of the matrices [ΔA_{λ}] and [Δα′]⁻¹ and the parameters Ψ_{Hb} and Ψ_{HbO2} represent the product of the matrices [ΔE′_{λ},] and [Δα′]⁻¹. To determine the level of cerebral tissue blood oxygen saturation (SnO₂), Equation 12 is rearranged using the form of Equation 1 and is expressed as follows:

$$Sn {O}_{2} \% = \frac{( {A}_{{HbO}_{2}} - {Ψ}_{{HbO}_{2}} )}{( {A}_{{HbO}_{2}} - {Ψ}_{{HbO}_{2}} + {A}_{Hb} - {Ψ}_{Hb} )} * 100 \% \qquad ( Eqn . 13 )$$

Note that tissue blood oxygen saturation is sometimes symbolized as StO₂, SctO2, CrSO₂, or rSO₂. The effective pathlength L_{b} cancels out in the manipulation from Equation 12 to Equation 13.

The value for SnO₂ is initially determined from an empirical reference of weighted combination of venous and arterial oxygen saturation (SmvO₂) value, for example using:

SmvO₂=Kv*SvO₂+Ka*SaO₂ (Eqn.14),

and the empirically determined values for SvO₂ and SaO₂, where the term “SvO₂” represents venous oxygen saturation, the term “SaO₂” represents arterial oxygen saturation, and the terms Kv and Ka are the weighted venous and arterial contributions respectively (Kv+Ka=1). The empirically determined values for SvO₂ and SaO₂ are based on data developed by discrete sampling or continuous monitoring of the subject's blood performed at or about the same time as the sensing of the tissue with the sensor; e.g., blood samples discretely collected can be analyzed by blood gas analysis and blood samples continuously monitored can be analyzed using a fiber optic catheter inserted within a blood vessel. The temporal and physical proximity of the NIRS sensing and the development of the empirical data helps assure accuracy. The initial values for Kv and Ka within Equation 14 are clinically reasonable values for the circumstances at hand. The values for A_{HbO2} and A_{Hb} are determined mathematically using the values for I_{bλ} and I_{xλ} for each wavelength sensed with the NIRS sensor (e.g., using Equation 3 & 4 for deep and shallow detectors or Equation 5 for a single detector). The calibration parameters Ψ_{Hb} and Ψ_{HbO2}, which account for energy losses due to scattering as well as other background absorption from biological compounds, are then determined using Equation 14 and non-linear regression techniques by correlation to different weighted values of SvO₂ and SaO₂; i.e., different values of Ka and Kv. Statistically acceptable values of Kv and Ka and Ψ_{Hb} and Ψ_{HbO2} are converged upon using the non-linear regression techniques. Experimental findings show that with proper selection of Ka and Kv, the calibration parameters Ψ_{Hb} and Ψ_{HbO2} are constant within a statistically acceptable margin of error for an individual NIRS sensor used to monitor brain oxygenation on different human subjects.

The above-identified process produces a NIRS sensor calibrated relative to a particular subject using invasive techniques, or a NIRS sensor calibrated relative to an already calibrated sensor (or relative to a phantom sample). When these calibrated sensors are used thereafter on a different subject, they do not account for the specific physical characteristics of the particular subject being tested. The present method and apparatus as described below permits a NIRS sensor to be calibrated in a non-invasive manner that accounts for specific physical characteristics of the particular subject being sensed.

One of the physical characteristics considered for calibration purposes by the present method and apparatus is the physical development stage of the subject. The accuracy of data (e.g., oxygen saturation level) produced by prior art NIRS sensors can vary in relationship to the physical development of the subject, and in particular the physical development of the subject's head and brain. As a result, when monitoring pediatric subjects, prior art NIRS sensors may be acceptably accurate for a first portion of the range of subject physical development characteristics, but may be less accurate over other portions of the wide range of physical development characteristics. Sensor inaccuracy can be partly a function of the variability of light signal depth of penetration over the range of pediatric subjects. For example, the light signal depth of penetration will vary for a given sensor configuration as a function of the physical characteristics (e.g., age, weight, brain development, head size) of the range of subjects. The depth of penetration is significant because, for example, light passing through white brain matter (which is disposed in a region inside of the region where gray brain matter resides) has different light absorption and light scattering characteristics than gray brain matter. A sensor that does not consider physical characteristics (e.g., age, weight, brain development, head size) of the subject, will not distinguish between those applications where both gray and white brain matter are interrogated, and those applications wherein only gray brain matter is interrogated.

To overcome this problem, the present apparatus and method accounts for physical characteristics of a subject including, but not limited to, one or more of the following: subject age, brain development, weight, and head size (e.g., measured by circumference). These physical characteristics rapidly change in pediatric subjects over time. Once growth and development rates of change decrease (e.g., when a pediatric subject reaches adolescence), the need to account for the change in the aforesaid subject characteristics also decreases. Brain development of a pediatric subject (as determined by age from birth and/or gestational age) can influence the background light absorption and scattering properties that may otherwise be constant in older subjects. Head characteristics such as skull thickness and/or brain gray matter thickness, (e.g., directly determined by CT or MRI imaging of the head or indirectly determined by head circumference) can affect the average light path between light source and detector(s) of an NIRS sensor, resulting in an inaccurate brain oxygenation measurement. The subject's weight, which typically relates to the head size and brain development, including gray and white matter thickness of a normally developing subject, can be used alone as a basis for calibrating a NIRS sensor. In some embodiments, more than one physical characteristic (e.g., weight and age) can be used to calibrate a NIRS sensor. Calibration based on more than one physical characteristic is particularly effective for abnormally developing subjects.

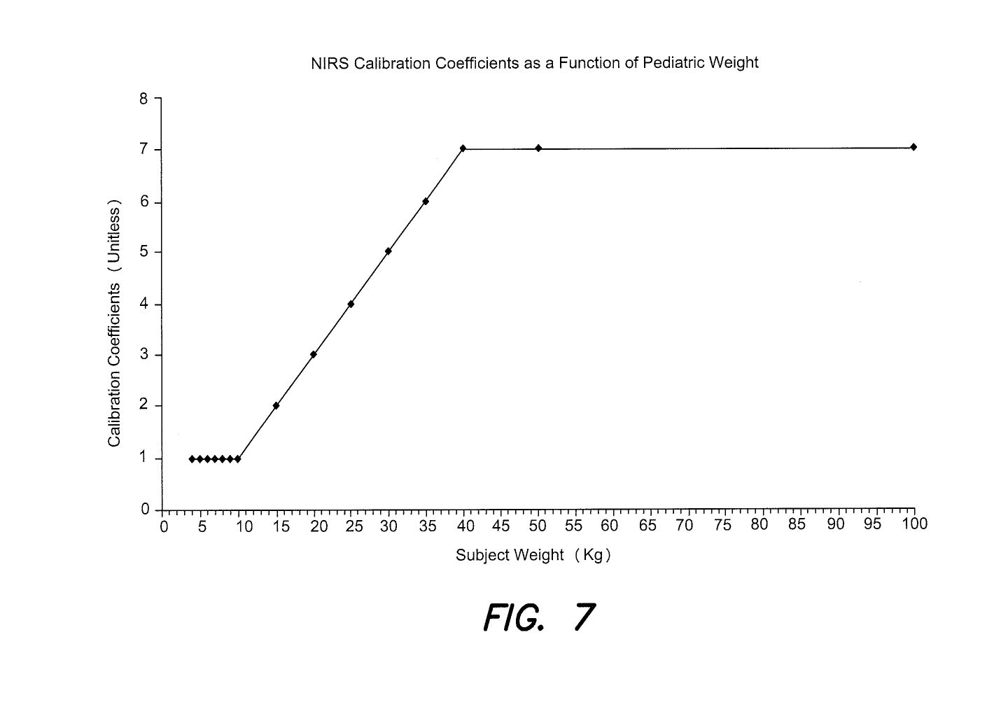

In some embodiments, the physical characteristics of pediatric subjects (e.g., age, weight, brain development, and head size) are incorporated via calibration constants within the above described algorithm (or other algorithm). An example of an algorithm with calibration constants representing subject weight is as follows:

$$Sct {O}_{2} = \frac{( Hb {O}_{2} + Hb {O}_{2 cal ( WT )} )}{( Hb {O}_{2} + Hb {O}_{2 cal ( WT )} + Hb + {Hb}_{cal ( WT )} )} \qquad Eqn . 15$$

where HbO_{2cal(WT)} and Hb_{cal(WT)} are calibration constants representing the subject's weight. As indicated above, equation 15 is an example of an algorithm incorporating the calibration constants, and the present method is not limited to equation 15. These calibration constants are a function of characteristics such as the subject's age, weight, head circumference, brain development, etc. The relationship between calibration constants and a particular physical characteristic (e.g., weight, head circumference, age, etc) can be represented in a graph, a database structure, a mathematical relationship, or the like. FIG. 7, for example, graphically illustrates the relationship between calibration constant values and the weight of a pediatric subject. The calibration constant values are shown on the y-axis and the weight (in kg) of the subject is shown along the x-axis. The curve disposed within the graph may be based on empirical data collected from a statistically significant pool of pediatric subjects, or it can be based on a mathematical characterization of empirical or theoretical data. The oxygen saturation level of pediatric subjects below a threshold of about 12 years of age and/or about 40 kilograms trends differently than that of subjects above the aforesaid threshold. FIG. 7 schematically shows a sloped linear relationship between the calibration constant values and the subject's weight. At a subject weight of about forty kilograms (40 kgs), the slope of the curve approaches a constant calibration constant value. Above the threshold (e.g., 40 kgs), therefore, the influence of pediatric physical characteristics becomes substantially linear and the subject's physical characteristics can be considered along with an adult model as is disclosed below. The implementation of calibration constants accounting for pediatric physical characteristics is accomplished by the operator of the NIRS device inputting the physical characteristic (e.g., the subject's weight, age, head circumference, etc) into the NIRS device.

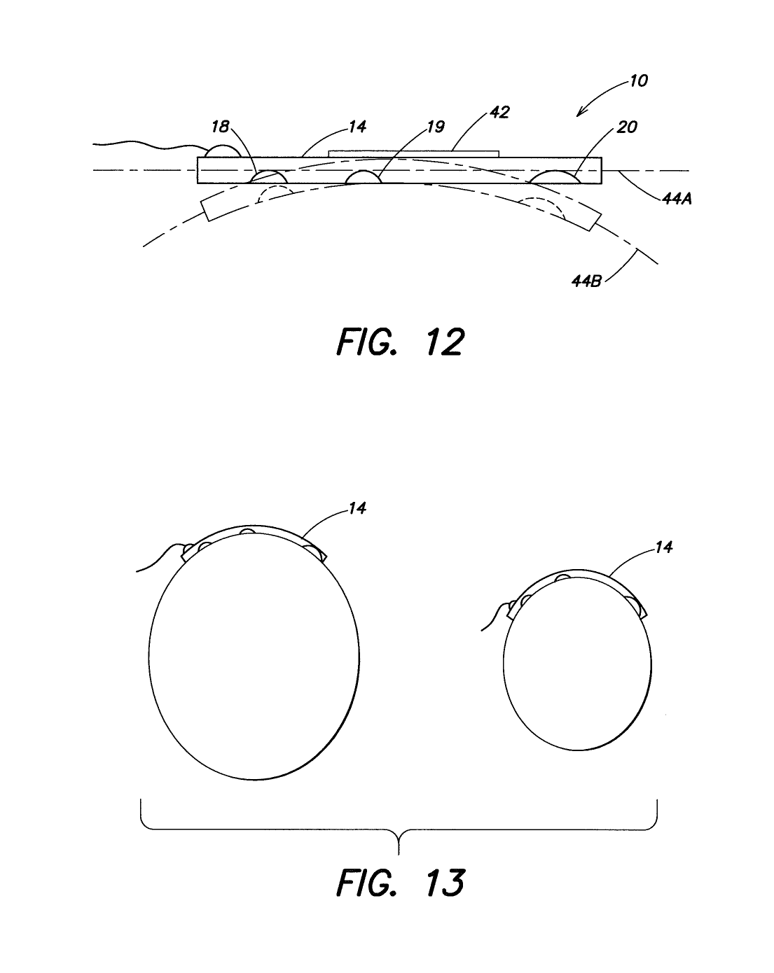

In some embodiments, the transducer portion 10 of the sensor includes a deflection sensor 42 operable to detect the amount of flexure of the assembly housing 14, which flexure can be used to provide information relating to the shape of the subject's head; e.g., the circumference of the subject's head. FIG. 12 schematically illustrates an assembly housing 14 disposed in a planar, flat position where a lengthwise centerline 44A of the housing 14 is a straight line. FIG. 12 also shows the housing 14 (shown in phantom) in a flexed position, wherein the lengthwise centerline 44B is disposed in a curvilinear configuration, deflected away from the straight line 44A. In the flexed position shown, the separation distance between the light source 18 and the detectors 19, 20 is less than the distance between the same in the planar position. FIG. 13 illustrates the same size transducer housing 14 shown on two different size 60 degree ellipses, which ellipses diagrammatically represent a human head. The effect of the different “head” sizes on the curvature of the mounted transducer housing 14 is readily apparent from these diagrams. An example of an acceptable deflection sensor 42 is an electrical strain gauge mounted relative to the assembly housing 14. The resistance of the strain gauge will change as a function of sensor bending. Connected to appropriate circuitry (e.g., electronic components on a flex circuit), the deflection sensor 42 relates the amount of deflection away from a planar position (i.e., the amount of flexure) in the form of a signal. The amount of deflection can, in turn, provide information relating to the shape and size of the subject's head. The signal from the transducer 10 is provided to the processor portion 12 of the sensor, where it is considered within an algorithm. While information relating to sensor deflection can be used alone as a surrogate to head size, shape and circumference, in some instances, the physical information determined from the flexure of the assembly housing 14 can be utilized along with one or more other physical characteristics of the subject within the algorithms. In other instances, the physical information determined from the flexure of the assembly housing 14 may be sufficient by itself, and therefore can be used alone within the algorithms The deflection sensor 42 is described above in tetras of an electrical strain gauge. The present invention is not limited to this embodiment, and in alternative embodiments may utilize other structure (e.g., piezoelectric sensors, fiber bragg grating sensors, etc.) operable to determine the magnitude of flexure of the transducer housing 14.

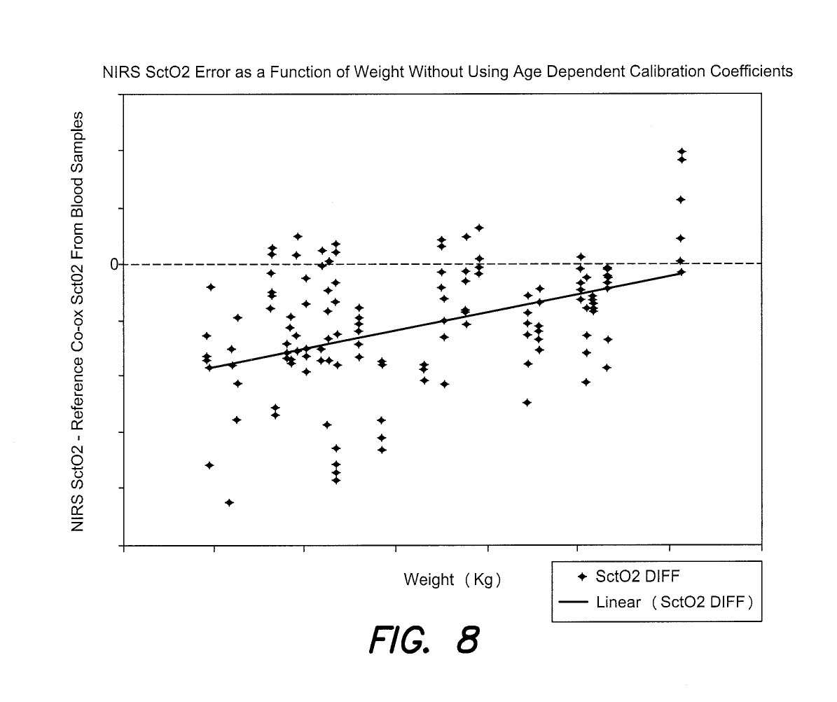

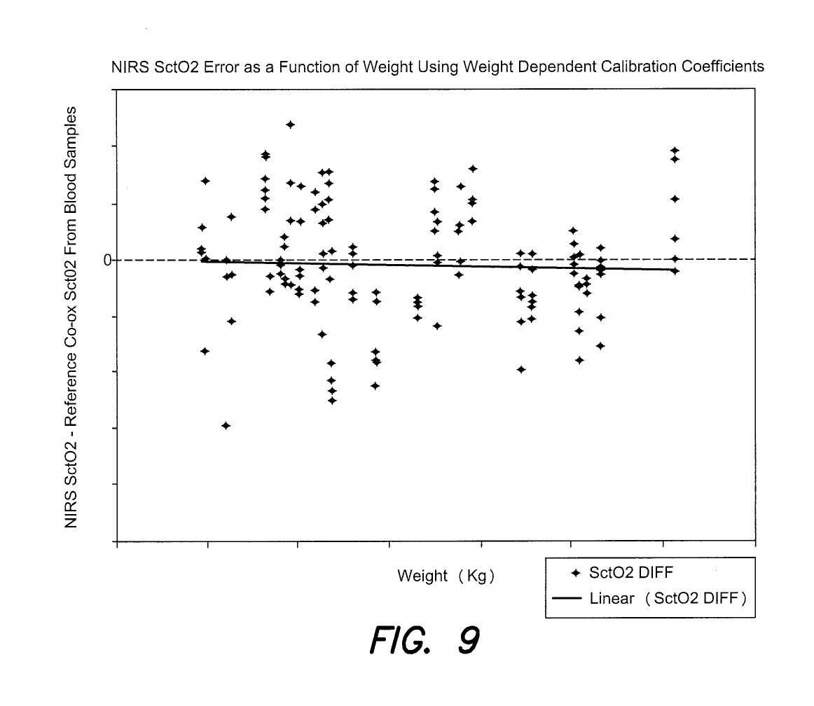

FIG. 8 graphically illustrates the blood oxygen saturation level (SctO₂) data as a function of subject weight. Specifically, the y-axis of the graph in FIG. 8 is a difference in SctO2 value determined by a NIRS sensor that does not account for the weight of a pediatric subject, and a SctO2 value invasively determined from blood samples from the same subject. The slope of the line representing the median value approaches zero difference as the weight of the subject approaches 35 Kg. FIG. 9 also graphically illustrates blood oxygen saturation level (SctO₂) data as a function of subject weight. In FIG. 9, however, the y-axis of the graph is a difference in SctO2 value determined by a NIRS sensor that does account for the weight of a pediatric subject, and a SctO2 value invasively determined from blood samples from the same subject. Comparing FIGS. 8 and 9, it can be seen that the accuracy of the NIRS sensor is improved over the range of pediatric subject weights, when the NIRS sensor algorithm accounts for the weight of the pediatric subject.

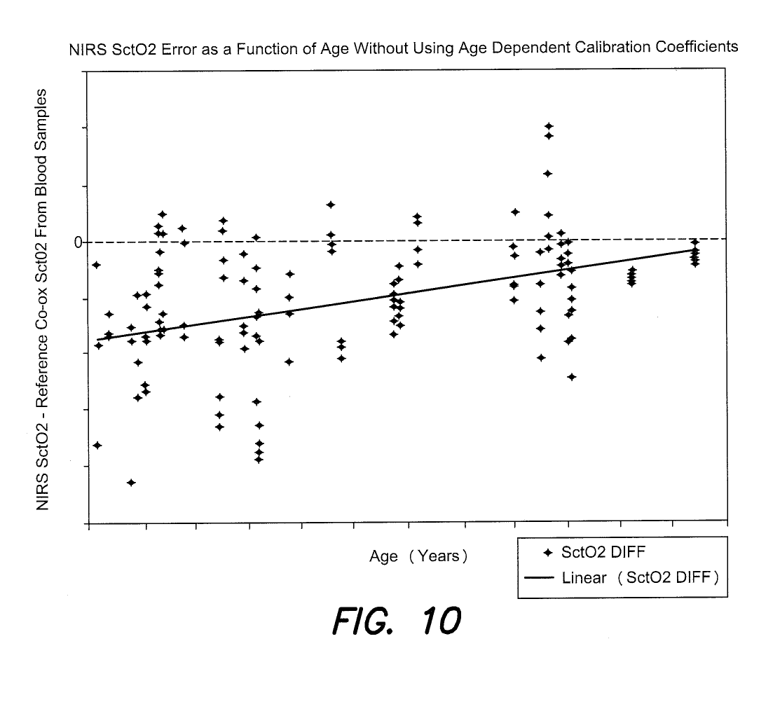

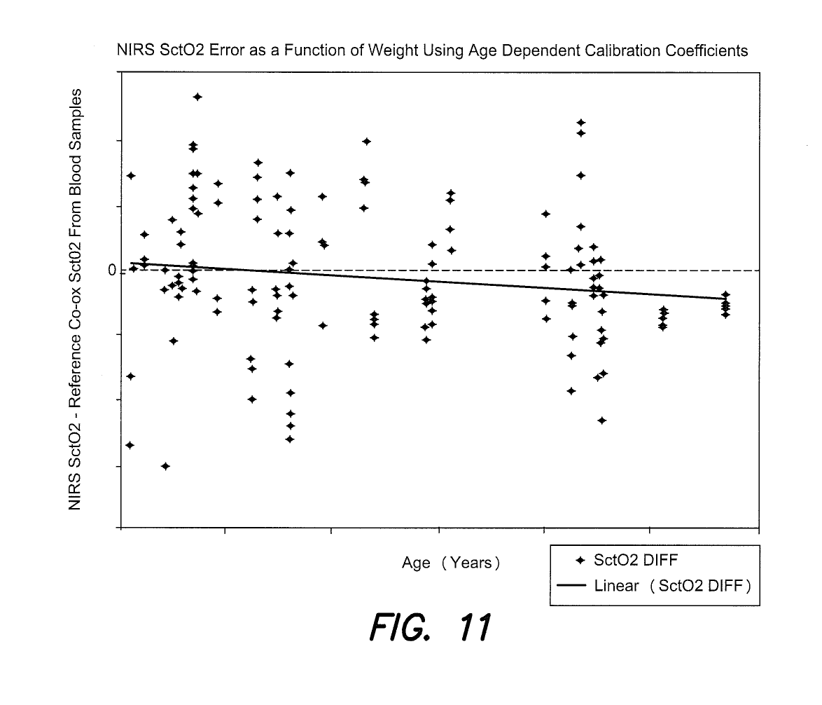

In a similar fashion, FIG. 10 graphically illustrates the blood oxygen saturation level (SctO₂) data as a function of pediatric subject age. Specifically, the y-axis of the graph in FIG. 10 is a difference in SctO2 value determined by a NIRS sensor that does not account for the age of a pediatric subject, and a SctO2 value invasively determined from blood samples from the same subject. The slope of the line representing the median value approaches zero difference as the age of the subject approaches twelve years old. In FIG. 11, the y-axis of the graph is a difference in SctO2 value determined by a NIRS sensor that does account for the age of a pediatric subject, and a SctO2 value invasively determined from blood samples from the same subject. Comparing FIGS. 10 and 11, it can be seen that the accuracy of the NIRS sensor is improved over the range of pediatric subject ages, when the NIRS sensor algorithm accounts for the age of the pediatric subject.

Certain physical characteristics of subjects will vary from subject to subject, such as but not limited to, tissue pigmentation and thickness and density of muscle and/or bone. The present method and apparatus accounts for background tissue's wavelength dependent light attenuation differences due to these subject-dependent physical characteristics by sensing the subject's tissue, creating signal data from the sensing, and using the signal data to create one or more “subject-specific” calibration constants that account for the specific characteristics of the subject. For example, during an initial phase of monitoring, light is transmitted into and sensed passing out of the subject's tissue. Signal data representative of the sensed light is analyzed to account for the physical characteristics of the subject, and one or more subject-specific calibration constants indicative of the specific physical characteristics are created. The subject-specific calibration constants are subsequently used to determine properties such as the blood oxygen saturation level, deoxyhemoglobin concentration, oxyhemoglobin concentration, etc.

The subject-specific calibration constants can be determined by using the sensed signal data to create a tissue optical property (TOP) index value. The TOP index value is derived from wavelength dependent light attenuation attributable to physical characteristics such as tissue pigmentation, thickness and density of tissue, etc. These physical characteristics are collectively considered in determining the TOP index value because the characteristics have absorption coefficients that increase with decreasing wavelength from the near-infrared region to the red region (i.e., from about 900 nm to about 400 nm) mainly due to the presence of melanin, the light absorbing pigmentation in skin and tissue. For example, it has been reported by S. L. Jacques et al., that light absorption in skin due to melanin can be described by the relationship: μₐ=1.70×10¹² (wavelength in nm)^{-3.48} [cm⁻¹] in the wavelength range from about 400 nm to about 850 nm. If the overall light absorption characteristics of tissue are modeled to follow that of melanin, then the TOP light absorption coefficients (α_{TOP}) can be determined using the same equation for the particular wavelengths of light used in the interrogation of the tissue (where A=1.7×10¹² and T=−3.48):

α_{TOP}=A*(wavelength)^{-T} (Eqn.15)

To determine the TOP index value, one or more of the wavelengths in the near-infrared region to the red region (i.e., from about 900 nm to about 600 nm; e.g., 690 nm, 780 nm, 805 nm, 850 nm) are sensed. Red wavelengths are favored because red light is more sensitive to the tissue optical properties than infrared light. Lower wavelengths of light could also be used, but suffer from increased attenuation from the higher tissue and hemoglobin absorption coefficients, resulting in reduced tissue penetration, reduced detected light signal strength, and resultant poor signal to noise ratio.

To calculate the TOP index value (identified in Equation 16 as “TOP”), a four wavelength, three unknown differential attenuation algorithm (following similarly to the derivation shown by Equations 3-10), is used such as that shown in Equation 16:

$$[ \begin{matrix}Δ {A}_{12}^{′} \\ Δ {A}_{13}^{′} \\ Δ {A}_{14}^{′}\end{matrix} ] {( {L}_{b} )}^{- 1} = [ \begin{matrix}Δ {α}_{r 12}^{′} & {Δα}_{o 12}^{′} & {Δα}_{TOP 12}^{′} \\ Δ {α}_{r 13}^{′} & {Δα}_{o 13}^{′} & {Δα}_{TOP 13}^{′} \\ {Δα}_{r 14}^{′} & {Δα}_{014}^{′} & {Δα}_{TOP 14}^{′}\end{matrix} ] [ \begin{matrix}Hb \\ Hb {O}_{2} \\ TOP\end{matrix} ] \qquad ( Eqn . 16 )$$

Alternatively, Equation 17 shown below could be used. Equation 17 accounts for energy losses “E” as described above:

$$[ \begin{matrix}Δ {A}_{12}^{′} & Δ {E}_{12}^{′} \\ Δ {A}_{13}^{′} & Δ {E}_{13}^{′} \\ Δ {A}_{14}^{′} & Δ {E}_{14}^{′}\end{matrix} ] {( {L}_{b} )}^{- 1} = [ \begin{matrix}Δ {α}_{r 12}^{′} & {Δα}_{o 12}^{′} & {Δα}_{TOP 12}^{′} \\ Δ {α}_{r 13}^{′} & {Δα}_{o 13}^{′} & {Δα}_{TOP 13}^{′} \\ {Δα}_{r 14}^{′} & {Δα}_{014}^{′} & {Δα}_{TOP 14}^{′}\end{matrix} ] [ \begin{matrix}Hb \\ Hb {O}_{2} \\ TOP\end{matrix} ] \qquad ( Eqn . 17 )$$

The TOP index value determinable from Equations 16 or 17 accounts for subject tissue optical properties variability and can be converted to a “corrective” factor used to determine accurate tissue blood oxygen saturation SnO₂. In some embodiments, the TOP index value can be used with a database to determine subject-specific calibration constants (e.g., Z_{Hb} and Z_{HbO2}). The database contains data, at least some of which is empirically collected, pertaining to oxyhemoglobin and deoxyhemoglobin concentrations for a plurality of subjects. The concentration data is organized relative to a range of TOP index values in a manner that enables the determination of the subject-specific calibration constants. The organization of the information within the database can be accomplished in a variety of different ways.

For example, the empirical database may be organized in the form of a graph having subject-specific calibration coefficients plotted along the y-axis versus TOP index values plotted along the x-axis. An example of such a graph is shown in FIG. 5, which contains data 30 representing the differences between calculated deoxyhemoglobin values (Hb) values and empirically derived deoxyhemoglobin values (the differences referred to in FIG. 5 as “Hb-offset2 data”), and a best fit curve 32 applied to a portion of that data 30. The graph also contains data 34 representing the differences between calculated oxyhemoglobin values (HbO2) values and empirically derived oxyhemoglobin values (the differences referred to in FIG. 5 as “Hb02-offset2 data”), and another best-fit curve 36 applied to a portion of that data 34. In the example shown in FIG. 5, a statistically significant number of the data 30, 34 for each curve lies within the sloped portion 32a, 36a (i.e., the portion that does not have a constant calibration constant value). At each end of the sloped portion 32a, 36a, the curves 32, 36 are depicted as having constant calibration values 32b, 32c, 36b, 36c for convenience sake. The values for the subject-specific calibration coefficients Z_{Hb} and Z_{HbO2} are determined by drawing a line (e.g., see phantom line 38) perpendicular to the TOP index value axis at the determined TOP index value. The subject-specific calibration constant (Z_{Hb}) for deoxyhemoglobin is equal to the value on the calibration constant axis aligned with the intersection point between the perpendicular line and the “Hb-offset2” curve, and the subject-specific calibration constant (Z_{HbO2}) for oxyhemoglobin is equal to the value on the calibration constant axis aligned with the intersection point with the “HbO2-offset2” curve”.

Alternatively, the subject-specific calibration constant values may be determined using an empirical database in a form other than a graph. For example, a mathematical solution can be implemented rather than the above-described graph. The mathematical solution may use linear equations representing the “Hb-offset2” and the “HbO2-offset2” curves.

Once the subject-specific calibration constant values are determined, they are utilized with a variation of Equation 13:

$$Sn {O}_{2} \% = \frac{( {A}_{Hb {O}_{2}} - {Ψ}_{{HbO}_{2}} + {Z}_{Hb {O}_{2}} )}{( {A}_{{HbO}_{2}} - {{Ψ}_{HbO}}_{2} + {Z}_{{HbO}_{2}} + {A}_{Hb} - {Ψ}_{Hb} + {Z}_{Hb} )} * 100 \% \qquad ( Eqn . 18 )$$

to determine the cerebral blood oxygen saturation level.

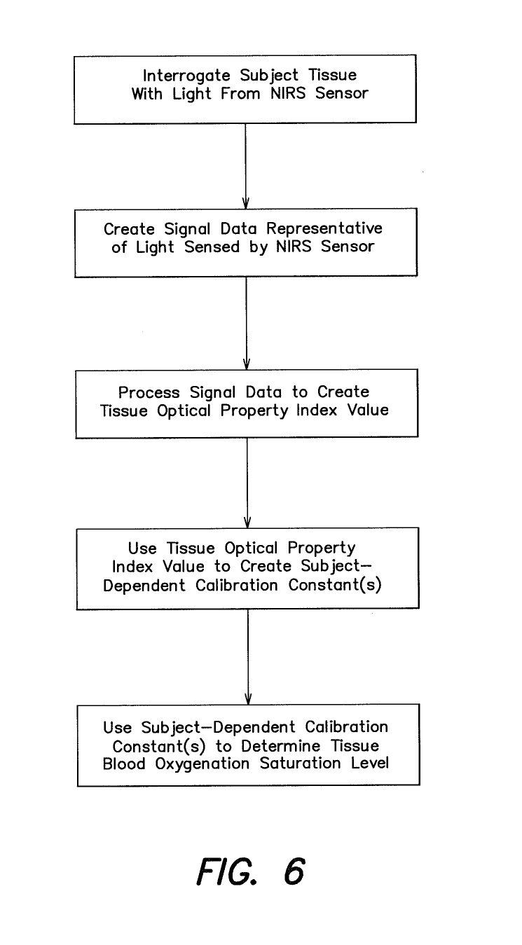

The above-described process for determining the subject-specific calibration constants can be performed one or more times in the initial period of sensing the subject to calibrate the sensor to that particular subject, preferably right after the sensor is attached to the subject. The subject-dependent calibration constants can then be used with an algorithm for measurement of a subject's blood oxygen saturation level using the same or different signal data. The algorithm in which the subject-dependent calibration constants are utilized may be the same algorithm as used to determine the constants, or a different algorithm for determining the tissue oxygen saturation level. For example, calibration constants can be used with the three wavelength method disclosed above in Equations 2-14, and in U.S. Pat. No. 6,456,862, which is hereby incorporated by reference. Prior to the cerebral blood oxygen saturation level being calculated, the subject-specific calibration constants Z_{Hb} and Z_{HbO2} can be incorporated as corrective factors into the three wavelength algorithm (e.g., incorporated into Eqn. 13). As a result, a more accurate determination of the subject's tissue oxygen saturation level is possible. FIG. 6 illustrates the above described steps within a flow chart.

In alternative embodiments, the TOP index methodology disclosed above can be used within an algorithm in a subject-independent manner. This approach does not provide all of the advantages of the above described subject-dependent methodology and apparatus, but does provide improved accuracy by specifically accounting for subject skin pigmentation. For example, the TOP absorption coefficients can be determined as described above and utilized within Equation 16 or Equation 17. Regardless of the equation used, the determined values for deoxyhemoglobin (Hb) and oxyhemoglobin (HbO₂) can subsequently be used to determine the tissue oxygen saturation level. For example, the Hb and HbO₂ values can be utilized within Equations 11 through 13.

Although the present method and apparatus are described above in terms of sensing blood oxygenation within cerebral tissue, the present method and apparatus are not limited to cerebral applications and can be used to determine tissue blood oxygenation saturation within tissue found elsewhere within the subject's body. If the present invention is utilized to determine the tissue blood oxygenation saturation percentage is typically symbolized as StO₂ or rSO₂.

Since many changes and variations of the disclosed embodiment of the invention may be made without departing from the inventive concept, it is not intended to limit the invention otherwise than as required by the appended claims.

What is claimed is:

1. A method for non-invasively determining a blood oxygenation level within a subject's tissue, comprising the steps of:
providing a spectrophotometric sensor operable to transmit light into the subject's tissue, and to sense the light;
inputting into the sensor at least one of the subject's age, weight, brain development, and head size;
spectrophotometrically sensing the subject's tissue along a plurality of wavelengths using the sensor, and producing signal data from sensing the subject's tissue; and
processing the signal data utilizing the at least one of the subject's age, weight, brain development, and head size, to determine the blood oxygen saturation level within the subject's tissue using a difference in attenuation between the wavelengths.

2. The method of claim 1, wherein the sensor includes a processor that is adapted to include one or more calibration constants that relate to subject age, weight, brain development, and head size.

3. The method of claim 2, wherein the processor is adapted to utilize one or more of a graph, a database structure, and a mathematical relationship to relate the one or more calibration constants to subject age, weight, brain development, and head size.

4. The method of claim 3, wherein the one or more of a graph, a database structure, and a mathematical relationship are based on empirically collected data.

5. An apparatus for non-invasively determining a blood oxygenation level within a subject's tissue, comprising:
a sensor having one or more transducer portions and a processor portion;
wherein each of the one or more transducer portions includes at least one light source and at least one light detector, and the light source is operable to transmit light along a plurality of wavelengths into the subject's tissue, and the light detector is operable to detect light along the wavelengths traveling through the subject's tissue, and each of the transducer portions is operable to produce signal data representative of the light sensed within the subject's tissue; and
wherein the processor portion is operably connected to the one or more transducer portions, and is adapted to receive input of at least one of the subject's age, weight, brain development, and head size, and the processor portion is adapted to process the signal data utilizing at least one of the subject's age, weight, brain development, and head size, to determine the blood oxygen saturation level within the subject's tissue using a difference in attenuation between the wavelengths.

6. The apparatus of claim 5, wherein the processor portion is adapted to include one or more calibration constants that relate to a subject age, weight, brain development, and head size.

7. The apparatus of claim 6, wherein the processor is adapted to utilize one or more of a graph, a database structure, and a mathematical relationship to relate the one or more calibration constants to a subject age, weight, brain development, and head size.

8. The apparatus of claim 7, wherein the one or more of a graph, a database structure, and a mathematical relationship are based on empirically collected data.

9. The apparatus of claim 5, wherein at least one of the transducer portions includes a housing to which the at least one light source and the at least one light detector are mounted and which housing has a lengthwise extending centerline and a deflection sensor adapted to sense flexure of the housing relative to the lengthwise extending centerline; and
wherein the processor portion is adapted to receive input from the deflection sensor and is adapted to process the signal data utilizing the deflection sensor input.

10. A method for non-invasively determining a blood oxygenation level within a subject's tissue, comprising the steps of:
providing a spectrophotometric sensor having one or more transducer portions and a processor portion, which transducer portions are operable to transmit light into the subject's tissue and sense light passing through the subject's tissue, and at least one of which transducer portions includes a housing having a lengthwise extending centerline and a deflection sensor adapted to sense flexure of the housing relative to the lengthwise extending centerline;
spectrophotometrically sensing the subject's tissue along a plurality of wavelengths using the transducer portions, and producing signal data from sensing the subject's tissue; and
processing the signal data, including using input from the deflection sensor to determine flexure of the at least one transducer portion, to determine the blood oxygen saturation level within the subject's tissue.

11. The method of claim 10, wherein the input from the deflection sensor is related to a physical characteristic of the subject during the processing of the signal data.

12. The method of claim 11, wherein the processing includes relating the input from the deflection sensor to at least one of a subject head size and subject head geometry.
